(19) United States
(12) Patent Application Publication
(10) Pub. No.: US 2025/0018107 A1
(43) Pub. Date: Jan. 16, 2025

(54) INFUSION DEVICES AND ASSOCIATED METHODS

(71) Applicant: Unomedical A/S, Lejre (DK)

(72) Inventors: Rachel Pytel, Newton, MA (US); Lisa Jaunet Van Kooten, Copenhagen (DK)

(21) Appl. No.: 18/902,739

(22) Filed: Sep. 30, 2024

Related U.S. Application Data
(63) Continuation of application No. PCT/EP2023/059057, filed on Apr. 5, 2023.
(60) Provisional application No. 63/328,806, filed on Apr. 8, 2022.

(30) Foreign Application Priority Data
May 23, 2022 (GB) 2207515.4

(51) Int. Cl. A61M 5/142

(52) U.S. Cl. CPC A61M 5/14248; A61M 2202/0007; A61M 2202/04; A61M 2205/0216; A61M 2205/0255; A61M 2205/0261; A61M 2205/10; A61M 2205/7527; A61M 2205/7545; A61M 2209/088; A61M 2210/04

(57) ABSTRACT

There is provided an infusion device for subcutaneous infusion of a therapeutic agent. The infusion device may comprise a cannula for insertion into a patient, and a filter disposed within the cannula, the filter comprising a filter material having a plurality of passageways extending therethrough to allow fluid flow through the filter and thereby through the cannula. The infusion device may have application in infusion pump systems such as an infusion pump for delivery of an insulin drug, the infusion pump system comprising a fluid pump and a reservoir, and an infusion set comprising the cannula and tubing for connecting the cannula to the reservoir.

### TECHNICAL FIELD

The invention relates to infusion devices and associated methods. Particularly, though not exclusively, the invention relates to infusion devices for infusing a therapeutic agent, such as insulin, to patients, such as those living with diabetes.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three to four times a day (depending on, inter alia, the type of diabetes and blood sugar levels of the patient). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and fewer needle sticks. The second mode is known as continuous subcutaneous insulin infusion (CSII) therapy.

Infusion pump systems for CSII therapy may be worn by the patient. The systems typically comprise a combined infusion pump and reservoir for containing an insulin drug, for example human insulin or analogue insulin, and an insulin infusion set. The infusion set may comprise a cannula (for example, a polymeric catheter or metal needle) for insertion subcutaneously into the patient and flexible tubing for fluidly connecting the cannula to the reservoir. Once the cannula is inserted into the patient, it may remain in place for a period of time, i.e. days, to allow for continuous delivery of the insulin drug. The current recommended wear time for insulin infusion sets is two to three days, to avoid problems that may arise relating to the infusion set itself or to the infusion site. However, such problems may still arise within recommended wear times, resulting in early removal of the infusion set and more frequent site rotation across infusion sites (for example buttocks, abdomen and arms).

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their infusion pumps, resulting in poorer patient outcomes.

It is known that problems at teh infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign material and attempt to degrade it, or otherwise encapsulate the material by forming a physical barrier to isolate it from the rest of the body. FBR is a problem for increasing wear times of infusion sets in CSII therapy, as the immune system reacts to the inserted cannula and the insulin drug. This limitation prevents realising the full potential of CSII therapy.

The problem presented by FBR is particularly acute in the development of so-called closed-loop systems. In closed-loop systems, am infusion pump system is used in combination with a continuous glucose monitoring (CGM) device, to continually monitor blood sugar levels and adjust the amount of insulin delivered to the patient automatically. CGM devices are already achieving 14-day wear times, and are thus currently achieving superior wear times compared to infusion sets.

Generally, unwanted species, whether biological, chemical or physical, present in infusible solutions have undesirable consequences for patients.

It is an object of embodiments of the invention to provide an improved infusion device that attempts to circumvent FBR, increase wear times of infusion sets, and/or at least mitigate one or more problems associated with known arrangements.

### SUMMARY OF THE INVENTION

According to an aspect of the invention, there is provided an infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path.

According to another aspect of the invention, there is provided infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path, wherein the filter material provides a physical filter medium for removing unwanted species by size exclusion, and wherein the filter is a modular filter comprising first and second sub-filters arranged to allow fluid flow therethrough in series.

According to another aspect of the invention, there is provided infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path, wherein the filter material provides a physical filter medium for removing unwanted species by size exclusion, and wherein the filter material provides a chemical filter medium for removing unwanted species by sorption, and wherein the filter is a modular filter comprising first and second sub-filters arranged to allow fluid flow therethrough in series.

According to another aspect of the invention, there is provided infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path, wherein the filter material provides a chemical filter medium for removing unwanted species by sorption, and wherein the filter is a modular filter comprising first and second sub-filters arranged to allow fluid flow therethrough in series.

According to another aspect of the invention, there is provided infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path, wherein the filter is a modular filter comprising first and second sub-filters arranged to allow fluid flow therethrough in series.

According to another aspect of the invention, there is provided infusion device for subcutaneous infusion of a therapeutic agent, the infusion device comprising: a flow path for conveying a therapeutic agent to patient; and a filter disposed within the flow path, the filter comprising a filter material having a plurality of passageways extending therethrough to allow fluid flow through the filter and thereby along the flow path, wherein the filter is a modular filter comprising first and second sub-filters arranged to allow fluid flow therethrough in series, and wherein the first sub-filter comprises the filter material and the second sub-filter comprises a further filter material different to the filter material.

According to a further aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula.

According to another aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula, wherein the filter material provides a physical filter medium for removing unwanted species by size exclusion, and wherein the filter is a modular filter comprising first and second subfilters arranged to allow fluid flow therethrough in series.

According to another aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula, wherein the filter material provides a physical filter medium for removing unwanted species by size exclusion, and wherein the filter material provides a chemical filter medium for removing unwanted species by sorption, and wherein the filter is a modular filter comprising first and second sub-filters arranged to allow fluid flow therethrough in series.

According to another aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula, wherein the filter material provides a chemical filter medium for removing unwanted species by sorption, and wherein the filter is a modular filter comprising first and second sub-filters arranged to allow fluid flow therethrough in series.

According to another aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula, wherein the filter is a modular filter comprising first and second sub-filters arranged to allow fluid flow therethrough in series.

According to another aspect of the invention, there is provided an infusion device for subcutaneous infusion of a therapeutic agent, the infusion device comprising: a cannula for insertion into a patient; and a filter disposed within the cannula, the filter comprising a filter material having a plurality of passageways extending therethrough to allow fluid flow through the filter and thereby through the cannula, wherein the filter is a modular filter comprising first and second sub-filters arranged to allow fluid flow therethrough in series, and wherein the first sub-filter comprises the filter material and the second sub-filter comprises a further filter material different to the filter material.

According to a further aspect of the invention, there is provided a method of subcutaneous infusion of a therapeutic agent, the method comprising the steps: providing an infusion device comprising a flow path for conveying a therapeutic agent to a patient, and a filter disposed within the flow path, the filter comprising a filter material having a plurality of passageways extending therethrough to allow fluid flow through the filter and thereby along the flow path; and delivering the therapeutic agent to an infusion site of a patient through the flow path so that the filter removes unwanted species from the therapeutic agent.

According to a further aspect of the invention, there is provided a method of subcutaneous infusion of a therapeutic agent, the method comprising the steps: of providing an infusion device comprising a cannula for insertion into a patient; and a filter disposed within the cannula, the filter comprising a filter material having a plurality of passageways extending therethrough to allow fluid flow through the filter and thereby through the cannula; inserting the cannula subcutaneously into an infusion site of a patient; and delivering the therapeutic agent to the infusion site through the cannula so that the filter removes unwanted species from the therapeutic agent proximate the point of delivery to the patient.

According to a further aspect of the invention there is provided a method of manufacturing an infusion device for subcutaneous infusion of a therapeutic agent, the method comprising the steps of: providing a cannula for insertion into a patient; and introducing a filter within the cannula, the filter comprising a filter material having a plurality of passageways extending therethrough to allow fluid flow through the filter and thereby through the cannula.

In certain embodiments, the filter material may provide a physical filter medium for removing unwanted species by size exclusion. Additionally, or alternatively, the filter material may provide a chemical filter medium for removing unwanted species by sorption.

In certain embodiments, the filter may be a modular filter comprising first and second sub-filters arranged to allow fluid flow therethrough in series. The first sub-filter may comprise the filter material and the second sub-filter comprises a further filter material different to the filter material. This difference may be in the type of material used, or the same material may be used with, for example, a different pore size.

In certain embodiments, the filter material (and/or the further filter material) may comprise a foam comprising a polyvinyl alcohol (PVA), a cellulose, a polyurethane

(PU), a polyester, a polyether and/or a collagen. Throughout this specification, discussion of the filter material is to be understood to apply equally to the further filter material. The passageways may be pores and the pores may have a pore size from 0.1 to 5 mm.

In certain embodiments, the filter material (and/or the further filter material) may comprise a zeolite. The passageways may be pores and the pores may have a pore size from 3 to 10 A.

In certain embodiments, the cannula may comprise a lumen wall having surface features for maintaining and/or locating the filter within the cannula. The cannula may be a soft polymeric catheter or a metal needle. The cannula may comprise one of a polytetrafluoroethylene (PTFE), a fluorinated ethylene propylene (FEP), a rubber, a polyethylene (PE), a polyurethane (PU) or a silicone material. The cannula may be insertable in the patient by an insertion needle.

In certain embodiments, the device may comprise tubing, and an infusion hub for maintaining the cannula in the patient and fluidly connecting the cannula to the tubing (i.e. the device may constitute an infusion set).

In certain embodiments, the device may comprise a fluid pump and a reservoir for storing a therapeutic agent, the reservoir being fluidly connected to the cannula, and the device may further comprise tubing fluidly connecting the cannula to the reservoir (i.e. the device may constitute an infusion pump system). The therapeutic agent may comprise insulin.

Devices and methods according to embodiments of the invention may be useful in inhibiting FBR at an infusion site, and thereby may avoid problematic occurrences such as coagulation, occlusion and/or inflammation at the infusion site, and/or encapsulation of the cannula. In particular, devices and methods according to embodiments of the
invention may be useful in inhibiting FBR at the infusion site in diabetic patients receiving CSII therapy, wherein the infusion site is a single infusion site in use for an extended period of time, for example at least four days.

Devices and methods according to embodiments of the invention may be useful in removing unwanted species from a therapeutic agent immediately before delivery of the therapeutic agent to a patient, for example where unwanted species include preservatives necessarily present in insulin solutions to stabilise and/or sterilise insulin solutions prior to delivery to a patient, but which are cytotoxic.

### BRIEF DESCRIPTION OF THE DRAWINGS

Embodiments of the invention will now be described, by way of example only, with reference to the accompanying figures, in which:

FIG. 1 is a schematic cross-sectional view of an infusion device according to an embodiment of the invention; and

FIG. 2 is a schematic view of an infusion device according to an embodiment of the invention.

### DETAILED DESCRIPTION OF EMBODIMENTS OF THE INVENTION

Embodiments of the invention have particular application for use with infusion pump systems such as an infusion pump for delivery of a therapeutic agent, the infusion pump comprising a fluid pump and a reservoir, and an infusion set having a cannula and tubing for connecting the cannula to the reservoir. However, other applications are contemplated, for example cannula access ports and alternative infusion pump systems such as patch pumps, which have no tubing. In certain embodiments, the infusion pump may be an insulin pump for CSII therapy and the therapeutic agent may be an insulin drug, for example human insulin or analogue insulin. Embodiments of the invention are intended for delivering insulin to a patient at a single infusion site over an extended period of time. Used herein, an extended period of time is to be understood to mean at least four days. More specifically, an extended period of time may include four to seven days, seven or more days, seven to 10 days, and 10 or more days. An extended period of time may include 14 or more days. Embodiments of the invention may be used in closed loop systems, wherein the extended period of time may be at least equal to the wear time and/or lifetime of a CGM device.

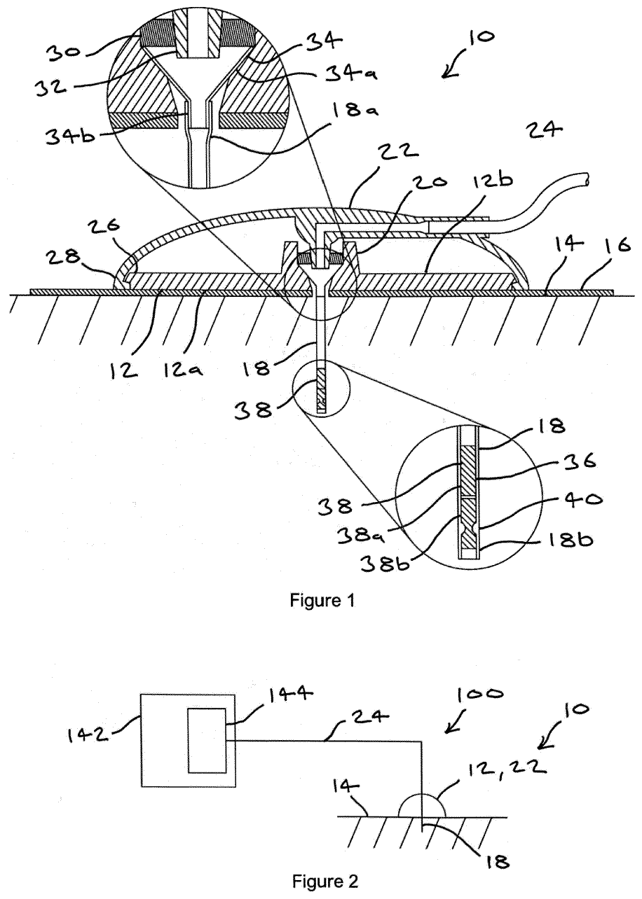

FIG. 1 illustrates an infusion device 10 according to an embodiment of the invention comprising components of an infusion set. The device 10 comprises a base 12 that is securable to the skin at an infusion site 14, for example by an adhesive patch 16, to maintain a cannula 18 within the sub-dermal fatty tissue of a patient. The base 12 has a first side 12a and an opposing second side 12b, the first side 12a being a patientfacing side or underside of the base 12 from which the cannula 18 extends. The base 12 further has a port 20 extending from the second side 12a for receiving the cannula 18. The base 12 is configured for attachment to an infusion cap 22 for delivering a therapeutic agent to the cannula 18 and, subsequently, into the infusion site 14. The therapeutic agent may comprise insulin. The therapeutic agent may be an insulin solution.

Together, the base 12 and the cap 22 provide an infusion hub, which serves as a fluid connector between the cannula 18 and a length of tubing 24. To effect and maintain attachment of the cap 22 to the base 12, the base 12 may have a rim 26 extending about its periphery that is configured to engage a cooperating rim 28 extending about the periphery of the infusion cap 22. The base 12 may comprise a self-sealing septum 30 disposed in or over the port 20 and generally made of a substantially resilient material biased toward a sealed position. Suitable materials for forming the septum 30 include a silicone or a polyurethane (PU). Securing the septum 30 within or to the port 20 may be effected by an adhesive, for example an epoxy adhesive or a cyanoacrylate adhesive. The septum 30 is configured to receive a port-engaging portion 32 of the infusion cap 22, to enable the fluid connection between the base 12 and the infusion cap 22. An insert 34 may be disposed within the port 20 to facilitate attachment and/or positioning of other components. In the illustrated example, the insert 34 has a frustoconical portion 34a and a cylindrical portion 34b.

The cannula 18 is a substantially tubular member for insertion in, and delivering the therapeutic agent to, the infusion site 14. A proximal end 18a of the cannula 18 is fluidly connected to a source of the therapeutic agent, for example a reservoir in an infusion pump system, via the tubing 24. An opposing, distal end 18b of the cannula 18 is positioned in the infusion site 14, extending to a desired depth to deliver the therapeutic agent. As the skilled person will appreciate, each of the base 12, the cannula 18, the infusion cap, and the tubing 24, in part provide a fluid pathway between the source of the therapeutic agent and the infusion site 14. The cannula 18 may provide a final component in the fluid pathway prior to the infusion site 14. As in the illustrated embodiment, the proximal end 18a of the cannula 18 may be secured to the cylindrical portion 34b of the insert 34 and the cannula-insert assembly may be secured to the base 12. In certain embodiments, the cannula 18 may be secured directly to the base 12. The arrangement and connection of the base 12 and the infusion cap 22 to one another may be provided by other suitable means known to those skilled in the art. A lumen 36, or bore, extends from the proximal end 18a to the distal end 18b for conveying the therapeutic agent through the cannula 18. The cannula 18 may be any suitable cannula, such as a polymeric catheter or metal needle. The cannula 18 may comprise any material suitable for implantation in a tissue site of a patient, for example a biocompatible polyurethane (PU). Those skilled in the art will understand that embodiments of the invention may be provided with cannulas having various lengths to accommodate various desired depths, including to accommodate patients with more or less sub-dermal fatty tissue. Suitable cannulas may have a length of from 5 to 50 mm and/or an inner diameter of 0.2 to 0.5 mm.

As above, the base 12 may be attachable to the skin by an adhesive patch 16, to maintain the cannula 18 within the infusion site 14. The adhesive patch 16 may extend across the entire underside of the base 12, to provide good adhesion to the skin. Moreover, suitable attachment of the base 12 to the skin may be provided by other means known to those skilled in the art.

The adhesive patch 16 may include a plurality of layers, for example a backing layer and an adhesive layer. The backing layer may be a non-woven PU film configured for comfortable attachment to the skin, for example the PU film may be suitably elastic to facilitate the patch 16 flexing with flexing of the skin. The adhesive layer comprises an adhesive for adhesion to the skin. Suitable adhesives include acrylic adhesives or soft silicone adhesives (SSA). Acrylic adhesives and SSAs are particularly suitable for attaching the infusion device 10 to the skin, as such adhesives may be non-sensitising, i.e. have good biocompatibility, and maintain adhesion in the presence of moisture, for example sweat, as well as at different temperatures. Moreover, such adhesives have good moisture and air permeability, and thereby may facilitate a long wear time of the infusion device 10. Other suitable adhesives include urethane-based adhesives and hydrocolloid adhesives. The adhesive is generally a pressure-sensitive adhesive.

The adhesive patch 16 may be secured to the first side 12a of the base 12 by an adhesive, for example an epoxy adhesive or a cyanoacrylate adhesive. In certain embodiments, the adhesive patch 16 may include a release layer, to protect it prior to use from dust, dirt and the like, which may reduce the efficacy of the adhesive patch 16 in adhering to the skin and/or increase the risk of infection and/or FBR at the infusion site. The release layer may be a silicone coated paper.

The tubing 24 may be made to have kink resistant and/or preservative retention properties. The tubing 24 may comprise a plurality of layers, for example two or three layers, which may include a tie layer. Suitable materials for the tubing 24 include thermoplastic polyurethane (TPU), which may provide an external layer, low density polyethylene (LDPE) and polypropylene (PP), either of which may provide an inert inner layer for contacting the therapeutic agent, and ethylene vinyl acetate (EVA), which may provide the tie layer.

Adhesives for use in embodiments of the invention are known in the art, and examples are disclosed in earlier patent applications, including WO2015/095639, US2016/0067106 and US2019/381281, the contents of which are incorporated herein by reference.

The device 10 may also comprise an insertion needle, for example when the cannula 18 is a soft, polymeric catheter. To this end, the device 10 may be configured to be applied to the infusion site 14 using an inserter device. Inserter devices, which are sometimes called inserters or injectors, are used commonly in the medical field for inserting medical devices subcutaneously into a patient.

Infusion sets, infusions hubs and inserter devices suitable for use in embodiments of the invention are known in the art, and examples are disclosed in earlier patent applications, including WO2007/092210, WO2010/112521, U.S. Pat. No. 755,173, U.S. Pat. Nos. 5,176,662, 5,257,980 and WO98/9856693, the contents of which are incorporated herein by reference.

The device 10 comprises filter 38. The filter 38 is intended to remove, for example by filtration, unwanted species present in the therapeutic agent to circumvent FBR. Used herein, unwanted species is to be understood to mean one or more species which may be present in the therapeutic agent, for example by design or accident, and which may be undesirable to remain in the therapeutic agent at the point of delivery to the infusion site. In particular, the filter 38 may remove unwanted species that occur in insulin solutions. Such unwanted species may be particulate and/or molecular in nature. Examples of particulate unwanted species include plastic particles, dust and insulin agglomerates, which have been produced during manufacture, storage, sterilization, or handling of the device 10 and/or the insulin solution. Examples of molecular
unwanted species include preservatives commonly used in insulin solutions, such as phenol, cresol (particularly m-cresol), benzyl alcohol, benzalkonium chloride, cetrimide, chlorobutanol, chlorhexidine, chlorocresol, hydroxybenzoates, phenethyl alcohol, phenoxyethanol and phenylmercuric nitrate.

In the illustrated embodiment, the filter 38 is disposed within the cannula 18. Locating the filter 38 within the cannula 18 allows the filter 38 to remove unwanted species from the therapeutic agent proximate the point of delivery to the patient, i.e. at or close to an end of the fluid pathway between the source of the therapeutic agent and the infusion site 14. Disposing the filter 38 within the cannula 18 is advantageous as it allows the filter 38 to remove unwanted species introduced to the therapeutic agent anywhere upstream of the point of delivery to the patient and/or unwanted species that are desirably present in the therapeutic agent up to the point of delivery, for example preservatives.

The filter 38 may comprise any filter material capable of removing one or more unwanted species from the therapeutic agent. To remove particulate unwanted species, the filter material may provide a physical filter medium for removing unwanted species by size exclusion, including whereby the filter material functions as a molecular sieve. Additionally, or alternatively, to remove molecular unwanted species, the filter material may provide a chemical filter medium for removing unwanted species by sorption, for example by adsorption or ion exchange, whereby the filter material binds with the molecular unwanted species to retain them within the filter 38. The filter material has a plurality of passageways, for example pores (i.e. interconnected hollow voids), extending therethrough to allow fluid flow through the filter 38.

The filter 38 may be a modular filter comprising first and second sub-filters 38a, 38b arranged to allow fluid flow therethrough in series for progressively removing different unwanted species from the therapeutic agent, for example unwanted species of varying sizes and/or varying molecular composition. Accordingly, the first sub-filter 38a may comprise a filter material different to that of the second sub-filter 38b. The filter 38 may similarly comprise a third, a fourth and so on sub-filters. Typically, the filter 38 forms a layer within the cannula 18 of a certain thickness, for example from 1 to 100 mm thick. Particularly suitable thicknesses include those from 2.5 to 5 mm thick. The device 10 may include a mesh and/or a membrane to maintain and/or locate the filter 38 within the cannula 18 (i.e. within the lumen 36). Suitable materials for the mesh and/or membrane may be silicone. In certain embodiments, the filter 18 may be attached to the wall of the lumen 36 by an adhesive, for example an epoxy adhesive or a cyanoacrylate adhesive. The filter 38 may be oversized to form a friction fit within the cannula 18. The cannula 18 may comprise surface features 40 on/within the inner wall of the lumen 36 to facilitate maintaining and/or locating the filter 38 within the cannula 18, for example one or more of bumps, constrictions and ridges, all of which may extend around an inner circumference of the lumen 36. The filter 38 may be disposed proximate the distal end 18b of the cannula 18.

Suitable filter materials include a foam, for example a polyvinyl alcohol (PVA) foam, and foams made of a cellulose, a polyurethane, a polyester, a polyether, a collagen or the like. The foam comprises a plurality of passageways in the form of interconnected pores extending therethrough to allow fluid flow through the filter 38. The foam may be any foam capable of removing particulate unwanted species from an insulin solution. The foam may remove particulate unwanted species from an insulin solution by a size exclusion process. The pores may be millipores (pore size from 0.1 to 100 mm). Suitable foams may include those having a pore size from 0.1 to 5 mm. Particularly suitable foams include those having a pore size from 0.3 to 1 mm. Additionally, or alternatively, suitable foams may include those having a porosity from 50% to 95% and/or dry density of from 0.1 to 1.5 grams per cubic inch. Particularly suitable foams include those having a porosity form 90% to 95%, and/or a for example a dry density of from 0.8 to 1.5 grams per cubic inch. The foam may absorb an aqueous solution such that the foam is saturated by at least 95% in a time from 0.1 to 1 minutes, for example from 3 to 30 seconds. The foam may have an ability to retain a liquid insulin solution such that the weight of the retained insulin solution is from 5 to 100 times, for example from 10 to 25 times, the weight of the foam material in the absence of an insulin solution. The foam may be crosslinked.

Particularly suitable filter materials include polyvinyl alcohol (PVA) foams, examples of which and methods of making are known in the art, and examples are disclosed in earlier patent applications, including U.S. Pat. Nos. 4,083,906 and 11,197,949, the contents of which are incorporated herein by reference.

Suitable filter materials include a zeolite. Zeolites are microporous, aluminosilicate minerals commonly used as commercial adsorbents and catalysts. The zeolite may be any zeolite capable of removing particulate and/or molecular unwanted species from an insulin solution. Most suitably, the zeolite is capable of removing phenol and/or cresol (particularly m-cresol) from an insulin solution. Particulate unwanted species may be removed from an insulin solution by a size exclusion process whereby the zeolite functions as a molecular sieve. Molecular unwanted species may be removed from the insulin infusion mixture by an adsorption process whereby the zeolite binds to the molecular components and retains them within the zeolite framework. Suitable zeolites include those having a pore size from 3 to 10 A. Particularly suitable zeolites are those having a pore size from 5 to 8 A. The zeolite may have a FAU, MOR or MFI framework type. The zeolite may have been thermally treated prior to being incorporated into the embodiments of the invention. For example, the zeolite may have been heated to a temperature in excess of 200° C., or even in excess of 700° C., prior to being incorporated into the embodiments of the invention. The zeolite may be coated, for example with a dextran. The zeolite may be a hydrophobic zeolite.

Particularly suitable zeolites include zeolite Y, mordenite and silicalite, for which example methods of preparation are known in the art, and disclosed in “Phenolic Preservative Removal from Commercial Insulin Formulations Reduces Tissue Inflammation while Maintaining Euglycemia”, Adam Mulka et al., ACS Pharmacology &
Translational Science 2021 4 (3), 1161-1174, the contents of which is incorporated herein by reference.

Suitable filter materials include an ion-exchange resin, including functionalised porous or gel polymers, which may remove unwanted species from an insulin solution by a gel permeation chromatography process. Moreover, gel polymers may be used to coat passageways in the filter material. A mesh and/or a membrane may be required to maintain a gel polymer in the cannula 18.

Generally, the filter material may be selected to have at least one material property that may facilitate the infusion of insulin at a single infusion site over an extended period of time, and thereby increase wear times, for example at least four days, including four to seven days, seven or more days, seven to 10 days, 10 or more days, and 14 or more days.

FIG. 2 illustrates an infusion device 100 according to an embodiment of the invention comprising components of an infusion pump system. The device 100 comprises the device 10 described above with reference to FIG. 1, including the base 12 and infusion cap 22, together forming the infusion hub, the cannula 18 and the tubing 24. The infusion hub, the cannula 18 and the tubing 24 together form an infusion set. The device 100 further comprises a combined infusion pump 142 and reservoir 144 for containing the therapeutic agent. A fluid pathway is provided from the reservoir 144 to the infusion site 14, for conveying the therapeutic agent. The fluid pathway extends from the reservoir 144 to the tubing 24, through the tubing 24, from the tubing 24 to infusion cap 22, through the infusion cap 22, from the infusion cap 22 to the base 12, through the base 12, from the base 12 to the cannula 18 and through the cannula 18.

The invention is not restricted to the details of any foregoing embodiments. For example, the filter 38 may be disposed anywhere within the fluid pathway, including in connectors that may be provided between the components described above with
reference to FIG. 2. The filter 38 may be in the base 12. The filter 38 may be disposed within the port 20. The filter 38 may be disposed within the insert 34, and may be disposed within the cylindrical portion 34b of the insert 34. In certain embodiments, the device 10 may consist of the cannula 18 and the filter 38 disposed therein, or the cannula 18 and the insert 34 having the filter disposed within the insert 34. In certain embodiments, the filter 38 may be provided by a mesh outside of cannula 18 to remove unwanted species from the therapeutic agent, the mesh being within the fluid pathway. Embodiments of the invention may comprise a plurality of filters, each of which may be as described above. Each of the plurality of filters may be spaced apart within the fluid pathway, such as a first filter disposed within the base 12 or the tubing 24, and a second filter disposed within the cannula 18. In certain embodiments, the filter material may provide the cannula 18, for example the distal end 18b of the cannula 18 may provide a tip comprising a ceramic filter material.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention extends to any novel one, or any novel
combination, of the features disclosed in this specification (including any accompanying claims, abstract and drawings), or to any novel one, or any novel combination, of the steps of any method or process so disclosed.

Attention is directed to all papers and documents which are filed concurrently with or previous to this specification in connection with this application and which are open to public inspection with this specification, and the contents of all such papers and documents are incorporated herein by reference.

1. An infusion device for subcutaneous infusion of a therapeutic agent, the infusion device comprising:
a flow path for conveying a therapeutic agent to patient; and
a filter disposed within the flow path, the filter comprising a filter material having a plurality of passageways extending therethrough to allow fluid flow through the filter and thereby along the flow path.

2. An infusion device for subcutaneous infusion of a therapeutic agent, the infusion device comprising:
a cannula for insertion into a patient; and
a filter disposed within the cannula, the filter comprising a filter material having a plurality of passageways extending therethrough to allow fluid flow through the filter and thereby through the cannula.

3. An infusion device according to claim 1 or 2, wherein the filter material provides a physical filter medium for removing unwanted species by size exclusion.

4. An infusion device according to claim 1, 2 or 3, wherein the filter material provides a chemical filter medium for removing unwanted species by sorption.

5. An infusion device according to any preceding claim, wherein the filter is a modular filter comprising first and second sub-filters arranged to allow fluid flow therethrough in series.

6. An infusion device according to claim 5, wherein the first sub-filter comprises the filter material and the second sub-filter comprises a further filter material different to the filter material.

7. An infusion device according to any preceding claim, wherein the filter material (and/or the further filter material) comprises a foam comprising a polyvinyl alcohol (PVA), a cellulose, a polyurethane (PU), a polyester, a polyether, and/or a collagen.

8. An infusion device according to any preceding claim, wherein the filter material (and/or the further filter material) comprises a zeolite.

9. An infusion device according to any preceding claim, wherein the passageways are pores and the pores have a pore size from 0.1 to 5 mm.

10. An infusion device according to any preceding claim, wherein the passageways are pores and the pores have a pore size from 3 to 10 A.

11. An infusion device according to any of claims 2 to 10, wherein the cannula comprises a lumen wall having surface features for maintaining and/or locating the filter within the cannula.

12. An infusion device according to any of claims 2 to 11, wherein the cannula is a soft polymeric catheter or a metal needle.

13. An infusion device according to any of claims 2 to 12, wherein the cannula comprises one of a polytetrafluoroethylene (PTFE), a fluorinated ethylene propylene (FEP), a rubber, a polyethylene (PE), a polyurethane (PU) and/or a silicone material.

14. An infusion device according to any of claims 2 to 13, wherein the cannula is insertable in the patient by an insertion needle.

15. An infusion device according to any of claims 2 to 14, wherein the device comprises tubing, and an infusion hub for maintaining the cannula in the patient and fluidly connecting the cannula to the tubing.

16. An infusion device according to any of claims 2 to 15, wherein the device comprises a fluid pump and a reservoir for storing a therapeutic agent, the reservoir being fluidly connected to the cannula.

17. An infusion device according to claim 16, wherein the device comprises tubing fluidly connecting the cannula to the reservoir.

18. An infusion device according to any preceding claim, wherein the therapeutic agent comprises insulin.

19. A method of subcutaneous infusion of a therapeutic agent, the method comprising the steps of:
providing the infusion device according to claim 1; and
delivering the therapeutic agent to an infusion site of a patient through the flow path so that the filter removes unwanted species from the therapeutic agent.

20. A method of subcutaneous infusion of a therapeutic agent, the method comprising the steps of:
providing the infusion device according to claim 2;
inserting the cannula subcutaneously into an infusion site of a patient; and delivering the therapeutic agent to the infusion site through the cannula so that the filter removes unwanted species from the therapeutic agent proximate the point of delivery to the patient.

21. A method according to claim 19 or 20, wherein the therapeutic agent comprises insulin.

22. A method of manufacturing an infusion device for subcutaneous infusion of a therapeutic agent, the method comprising the steps of:
providing a cannula for insertion into a patient; and
introducing a filter within the cannula, the filter comprising a filter material having a plurality of passageways extending therethrough to allow fluid flow through the filter and thereby through the cannula.
